(19) United States
(12) Patent Application Publication
(10) Pub. No.: US 2003/0119074 A1
(43) Pub. Date: Jun. 26, 2003

(54) Diagnosis and treatment of dementia utilizing thrombospondin

(76) Inventors: George Jackowski, Kettleby (CA); Rulin Zhang, Toronto (CA)

Correspondence Address: MCHALE & SLAVIN, 4440 PGA BLVD, SUITE 402, PALM BEACH GARDENS, FL, 33410

(21) Appl. No.: 10/032,229

(22) Filed: Dec. 20, 2001

(51) Int. Cl. G01N 33/53; G01N 33/567; G01N 33/542

(52) U.S. Cl. 435/7.9

(57) ABSTRACT

A method for diagnosing various forms of dementia, including MCI, and Alzheimer's disease(AD) is disclosed. The method involves directly detecting the presence of a biochemical marker, specifically thrombospondin, in bodily fluid, preferably blood or a blood product. The detection is by an immunoassay incorporating an antibody specific to thrombospondin, or alternatively an autoantibody to a thrombospondin antibody.

### FIELD OF THE INVENTION

The present invention relates to a method for the diagnosis and treatment of dementia utilizing thrombospondin; particularly to a process and device for quantifying the presence of thrombospondin, e.g. a central lab or point-of-care immunoassay, to wit a diagnostic kit, which utilizes antibodies to determine the presence of thrombospondin in circulating body fluids, thereby enabling a diagnosis of dementia, particularly Alzheimer's dementia. The invention further relates to processes for therapeutic intervention and therapeutic targets related thereto.

### BACKGROUND OF THE INVENTION

Cognitive impairment is a serious medical issue that is of increasing concern to society. It is crucial that treatment strategies are developed that effectively stop or reverse the declines associated with this disorder.

In accordance with accepted criteria, the diagnosis of dementia requires the presence of multiple cognitive deficits in addition to memory impairment. Early in the disease, memory impairment may be the only clinical finding, and this single finding would not meet the diagnostic criteria for dementia. To fulfill a diagnosis of dementia, cognitive impairment must be of the degree that social or occupational function is reduced, with the functional impairment representing a decrease in the patient's normal ability.

A variety of diagnostically oriented scales exist to define the degree of mental status and categorize a patient's condition based upon the degree of cognitive impairment. Tests such as the Short Portable Mental Status Questionnaire (SPMSQ), the Folstein Mini-Mental Status Examination (MMSE) or the Clinical Dementia Rating scale (CDR) usually identify cognitive impairment. The MMSE includes assessments of orientation, memory, attention and calculation, language, ability to follow commands, reading comprehension, ability to write a sentence and ability to copy a drawing. It is noted that education, occupation and cultural and background factors may often strongly influence MMSE scores. The CDR was designed to characterize subjects from normal function through various stages of dementia.

Age-related cognitive decline is characterized by memory loss without loss of other cognitive functions. A disorder similar to age-related cognitive decline is described as “mild cognitive disorder” in the World Health Organizations ICD-10 classification (International Statistical Classification of Diseases, 10th rev.). The diagnosis of mild cognitive disorder can be made if the cognitive decline is temporally related to cerebral or systemic disease. Age-related cognitive decline represents cognitive changes that are within normal limits given the person's age. Age-associated cognitive decline is characterized by a decline in only one of the five broad neuropsychologic domains associated with dementia: memory and learning; attention and concentration; thinking; language; and visuospatial functioning.

In accordance with findings of the International Psychogeriatric Association, additional criteria should be met to make a diagnosis of age-related cognitive decline. These criteria include the report of cognitive decline from a reliable source, a gradual onset of at least six months' duration and a score of more than one standard deviation below the norm on standardized neuropsychologic testing such as the MMSE.

The term “mild cognitive impairment” (MCI) describes a condition that may or may not eventually lead to dementia. At least one study indicates that patients with mild cognitive impairment exhibited a more rapid decline in cognitive function than control patients, albeit a less rapid decline than patients with mild Alzheimer's disease. Mild Cognitive Impairment is often characterized by mild recent memory loss without dementia or significant impairment of other cognitive functions to an extent that is beyond that expected for age or educational background.

The assumption of a relationship between Mild Cognitive Impairment and AD is based on physiological similarities. It has been reported that MCI patients often present with significant medical temporal lobe atrophy, while others have high cerebrospinal fluid and/or low CSF-ββ amyloid (Aββ) 42 concentrations, factors that are associated with the senile plaques common to AD. Furthermore, it has been reported that genetic similarities exist between the conditions. The strongest physiologic predictor of familial AD, for example, may be the presence of apolipoprotein E gene (ApoE), and the E4 allele is overrepresented in both AD and MCI patients. These characteristics, in combination with the fact that the onset of AD is insidious and has a course that is gradually progressive, has lead practitioners to believe that neuropathologies exist many years before any symptoms occur. If, in fact, MCI is an early sign of AD, then the accurate and early evaluation and treatment of MCI individuals might prevent further cognitive decline, including development of Alzheimer's disease.

It is apparent that the definitions of and the distinctions between mild cognitive disorder, age-associated cognitive decline and mild cognitive impairment remain extremely controversial. Nevertheless, an advisory panel to the US Food and Drug Administration recently decided that mild cognitive impairment, “a condition separate from Alzheimer's disease,” is a valid target for new drug therapies, regardless of whether a particular drug also slows the progression to dementia. Furthermore, the Peripheral and Central Nervous System Drugs Advisory Committee has stated that more than 80% of patients with mild cognitive impairment develop Alzheimer's disease within 10 years at a rate of 10% to 15% of patients per year. This finding will no doubt lead certain medical experts to view mild cognitive impairment as early Alzheimer's disease rather than a distinct condition.

The research literature suggests that many patients with MCI progress to AD. While figures vary as to the number of individuals with MCI who go on to develop AD, the percentage frequently seen in the literature is up to 40% in three years with a diagnosis of Mild Cognitive Impairment. Thus, treatment of MCI is of great interest to clinicians in that it may prevent, delay or even reverse disease-associated brain deterioration.

Alzheimer's disease, also referred to as Alzheimer's dementia or AD is a progressive neurodegenerative disorder that causes memory loss and serious mental deterioration. Diagnosticians have long sought a means to definitively identify AD during the lifetime of demented patients, as opposed to histopathological examination of brain tissue, which is the only present means available for rendering an ultimate diagnosis of AD. AD is the most common form of dementia, accounting for more than half of all dementias and affecting as many as 4 million Americans and nearly 15 million people worldwide. Dementia may start with slight memory loss and confusion, but advances with time reaching severe impairment of intellectual and social abilities. At age 65, the community prevalence of AD is between 1-2%. By age 75, the figure rises to 7%, and by age 85 it is 18%. The prevalence of dementia in all individuals over age 65 is 8%. Of those residing in institutions, the prevalence is about 50%, at any age.

The social impact of this disease is enormous, caused by the burden placed on caregivers, particularly in the latter stages of the disease. The substantial economic costs are largely related to supportive care and institutional admission. The rapidly increasing proportion of elderly people in society means that the number of individuals affected with AD will grow dramatically, therefore finding an early accurate diagnosis and a cure for AD is becoming an issue of major importance world wide.

When an individual is suspected of AD, several recommended tests are performed: (1) Mini Mental State Examination (MMSE) (as described above), (2) Laboratory tests—complete blood count, measurement of thyroid stimulating hormone, serum electrolytes, serum calcium and serum glucose levels, (3) Neuroimaging—most commonly used is computed tomography (CT) which has a role in detecting certain causes of dementia such as vascular dementia (VaD), tumor, normal pressure hydrocephalus or subdural hematoma. However, neuroimaging is less effective in distinguishing AD or other cortical dementias from normal aging. In primary care settings, some suggest that CT could be limited to atypical cases, but others recommend routine scanning. Magnetic resonance imaging (MRI) currently offers no advantage over CT in most cases of dementia.

While Alzheimer's is the most common form of dementia, accounting for at least 60% of cases, diagnostic procedures for determining the exact cause of dementia, among more than 80 different species, is difficult at best. Furthermore, the currently performed tests are inadequate in differentiating AD from other types of dementia.

In comparison to other disease areas, the field of dementia raises questions concerning the value of diagnosis, since there is often no specific cure or distinctly effective therapy available. While dementia related disorders, as outlined above, cannot be cured at present time, there does exist symptomatic treatment, e.g. drugs such as acetylcholinesterase inhibitors, which offer the hope of forestalling the progression of symptoms of MCI or AD and improvement of cognition and behavior are now licensed by the U.S. Food and Drug Administration. Other drugs are at different stages of clinical trials: (1) Drugs to prevent decline in AD—DESFERRIOXAMINE, ALCAR, anti-inflammatory drugs, antioxidants, estrogen, (2) Neurotrophic Factors: NGF, (3) Vaccine the recent most exciting report by Schenk et al. (Nature 1999;400:173-7) raises the hope of a vaccine for AD.

The specificity of the various therapies thus require sophisticated diagnostic methodologies, having a high degree of sensitivity for dementia, with particular attention being drawn to MCI and AD, in order to insure their success.

Although there are a multitude of tests available which aid in the diagnosis of AD, the only true existing diagnosis is made by pathologic examination of postmortem brain tissue in conjunction with a clinical history of dementia. This diagnosis is based on the presence in brain tissue of neurofibrillary tangles and of neuritic (senile) plaques, which have been correlated with clinical dementia. Neuritic plaques are made up of a normally harmless protein called amyloid-beta. Before neurons begin to die and symptoms develop, plaque deposits form between neurons early on in the disease process. The neurofibrillary tangles are interneuronal aggregates composed of normal and paired helical filaments and presumably consist of several different proteins. The internal support structure for brain neurons depends on the normal functioning of a protein called tau. In Alzheimer's disease, threads of tau protein undergo alterations that cause them to become twisted. The neurohistopathologic identification and counting of neuritic plaques and neurofibrillary tangles requires staining and microscopic examination of several brain sections. However, the results of this methodology can widely vary and is time-consuming and labor-intensive.

Given the ability of both current and prospective pharmacological therapies to forestall and/or reverse the onset and/or progress of various cognitive disorders, such as MCI, Alzheimer's dementia and the like, an early diagnosis of AD will assist to better manage the care of patients. There are many cases where non-AD dementia could be confused with AD dementia. Such examples include small, undetected strokes which temporarily interrupt blood flow to the brain. Clinically depressed patients or those with Parkinson's disease can also experience lapses in memory. Many older people are on a variety of medications which as a side effect may, alone or in conjunction, impair their ability to perform cognitive tasks.

Thus, if diagnostic techniques for the early differentiation of dementia, particularly AD could be provided, physician's would achieve an enhanced ability to prescribe appropriate therapeutic intervention at an early stage in the pathogenesis of this disease.

Various biochemical markers for AD are known and analytical techniques for the determination of such markers have been described in the art. As used herein the term “marker” “biochemical marker” or “marker protein” refers to any enzyme, protein, polypeptide, peptide, isomeric form thereof, immunologically detectable fragments thereof, or other molecule, whose presence, absence, or variance in circulating body fluids from so-called “normal” levels, are indicative of dementia. Most particularly, such markers may be illustrated as being released from the brain during the course of dementia related changes, e.g. AD pathogenesis. Such markers include, but are not limited to, any unique proteins or isoforms thereof that are particularly associated with the brain.

There are a number of different potential uses for biomarkers in evaluation of dementia, and each use could involve a different marker or set of markers. Such uses may include, but are not limited to, the use of a marker to distinguish AD or MCI from other causes of dementia; distinguishing dementia from the non-pathological effects of aging; monitoring the progress of the disease after clinical symptoms become apparent; utilization of a surrogate to monitor the efficacy of the forthcoming therapies for AD; and isolating markers which have utility as risk assessment factors for AD; and identifying both the earliest biological changes occurring in the brain and other changes that occur as the disease progresses.

Ideally, it would be preferable to isolate a single marker to fulfill all requirements with a high degree of sensitivity and specificity, however this may be an unreasonable goal. Any individual marker needs to be assessed by sensitivity, specificity, reliability and validity for the type of clinical situation to which it is meant to apply. A marker which is poor at distinguishing AD from other causes of dementia, could nevertheless be an excellent marker for monitoring the progression of the disease process or the response to therapy.

With regard to diagnostic devices, the clinical evaluation and use of point-of-care tests, as well as central laboratory tests utilizing biological markers are valuable tools for evaluating risk, monitoring disease progression and guiding therapeutic interventions. The advantages which flow from the use of biological markers as diagnostic tools include strengthening the certainty of the clinical diagnosis, distinguishing AD from other causes of dementia, and quantifying the severity of the disease and rate of progression. In addition, tests using biological markers should be rapid, non-invasive, simple to perform and inexpensive.

What is lacking in the art is a relatively non-invasive method and device therefore effective for definitively diagnosing various forms of dementia, particularly MCI and Alzheimer's dementia in living patients. Additionally, a definitive method of assessing the risk of developing AD is greatly needed.

### DESCRIPTION OF THE PRIOR ART

In U.S. Pat. No. 5,508,167, Roses et al. describe methods for diagnosing AD involving the detection of an apolipoprotein E type 4 (ApoE4) isoform or DNA encoding ApoE4. The methods can use blood samples and are analyzed by an immunochemical assay. The blood sample is optionally combined with a reducing agent to reduce the disulfide bond in cysteine residues to the corresponding reactive sulfhydryl groups. Roses et al. further describes a kit for detection of the ApoE4 isoform. The test is based on the differences in the amino acid sequences of the three major ApoE isoforms. The test is not specific for, nor is it suggestive of the use of thrombospondin as a marker for dementia.

Generally, most scientific papers tend to focus on the peptide, β-amyloid, since it is postulated to be a major determinant of AD. This is supported by the observation that certain forms of familial AD mutations result in the over production of β-amyloid, particularly the longer form (1-42) which aggregates more readily than the shorter form. Hensley et al. (Proc. Natl. Acad. Sci., (1994), 91, pp 3270-3274) examine the neurotoxicity based on free radical generation by the peptide β-amyloid in its aggregation state. Several synthetic fragments of the peptide are tested for resulting neurotoxicity. Based on the fact that oxygen seems to be a requirement for radical generation and glutamate synthetase and creatine kinase enzymes are oxidation-sensitive biomarkers, the inactivation of these enzymes are utilized as indicators of active attack on biological molecules by these fragmented β-amyloid aggregates. Buce et al, Department of Geriatrics and Adult Development, Mount Sinai School of Medicine, have conducted immunohistochemical localization of thrombospondin in normal human brains in the hippocampus and inferior temporal cortex. The distribution of thrombospondin staining in patients with Alzheimer's disease was found to be comparable to control subjects. However, in patients with Alzheimer's disease a subset of pyramidal neurons that may be vulnerable in Alzheimer's disease exhibited decreased staining. This decrease in the intensity of labeling was theorized as possibly targeting a neuronal population prone to early degeneration. In addition, thrombospondin staining was demonstrated in senile plaques in Alzheimer's disease. These results suggest that thrombospondin may be involved in the process of neuronal degeneration and senile plaque formation.

### SUMMARY OF THE INVENTION

The present invention relates to a method for the diagnosis of dementia, e.g. Alzheimer's dementia (AD), particularly to a method for diagnosing dementia by testing for the presence of thrombospondin in body fluids, particularly in blood, blood products, CSF, urine, saliva and the like. The invention further relates to a process for quantifying the presence of thrombospondin particularly as it relates to the diagnosis of Alzheimer's dementia. More particularly, the invention relates to an immunoassay technique which utilizes antibodies to enable the diagnosis of various forms of dementia, particularly Alzheimer's dementia, as evidenced by the presence of thrombospondin.

The present invention relates to the use of thrombospondin as a marker of dementia, particularly Alzheimer's dementia, methods for determining the presence of thrombospondin in body fluids, and a diagnostic device, e.g. an ELISA system for diagnosing, subtyping and monitoring Alzheimer's disease. The invention is based on the discovery that thrombospondin is released into the circulation, presumably from the brain, and can be detected in body fluids outside the brain in patients suffering from Alzheimer's disease.

Monoclonal or polyclonal antibodies which recognize various epitopes of thrombospondin can be used in immunoassays, wherein they enter into an immunoreaction which can be monitored and/or quantified to detect circulating thrombospondin proteins or the various isoforms, immunological fragments, etc., as herein described, which are indicative of a disease state in suspected individuals. Alternatively, the thrombospondin proteins themselves may be used in immunoassays to detect circulating autoantibodies in such individuals. The occurrence of Alzheimer's dementia is characterized by the recognition of levels of a particular biochemical marker in bodily fluid, said levels correlating to the manifestation of Alzheimer's dementia symptoms as quantified by MMSE testing.

As a risk assessment test, the recognition of levels of such markers which are indicative of the presence of MCI or related dementia, and which may reasonably be deemed to be a precursor to the development of Alzheimer's dementia further augments the diagnostic capability afforded to the skilled practitioner.

Accordingly, it is an objective of the instant invention to provide a relatively non-invasive and highly sensitive method for the definitive diagnosis of dementia, particularly MCI and Alzheimer's disease.

It is a further objective of the invention to provide a method which includes analysis of at least one body fluid of a patient to determine the presence of thrombospondin as an indicator of dementia, e.g. AD vs. other forms of cognitive disorders.

It is a still further objective of the instant invention to provide an immunoassay effective for the recognition of thrombospondin in one or more human body fluids.

It is a still further objective of the invention to provide a test kit for the diagnosis of dementia, e.g. MCI and AD comprising an immunoassay test, e.g. a point-of-care or central lab test, which is relatively non-invasive and which can be performed utilizing a sample comprising body fluids, e.g. blood or any blood products, CSF, urine, saliva and the like.

Other objects and advantages of this invention will become apparent from the following description taken in conjunction with the accompanying figures wherein are set forth, by way of illustration and example, certain embodiments of this invention. The figures constitute a part of this specification and include exemplary embodiments of the present invention and illustrate various objects and features thereof.

### BRIEF DESCRIPTION OF THE FIGURES

FIG. 1 is an analysis of thrombospondin levels in human sera enriched by heparin affinity column;

FIG. 2 is a Western blot analysis of thrombospondin levels in human sera of Alzheimer's patients and age-matched control.

### DETAILED DESCRIPTION OF THE INVENTION

Recently, a group at the University of Kentucky found that the process of amyloidosis can be inhibited by low molecular weight heparins. It is believed that these heparin molecules bind to heparin sulfate proteoglycans and slow down the process of amyloid formation. Previous reports have shown that highly sulfated glycosaminoglycans are present in all forms of amyloid identified, these molecules are a fundamental part of basement membrane structure and may provide an initiation point for amyloid fibrillogenesis.

With this in mind, the instant inventors set out to compare the various heparin-binding molecules present in both AD and control sera.

A protocol for fractionation and enrichment of heparin sulfate proteoglycans in human sera for biomarker discovery using immobilized heparin beads was determined. While not wishing to be bound to any particular theory, it was theorized that since heparin sulfate proteoglycans were found in amyloid deposits in amyloid diseases, that they may play an important role in fibril formation.

### Materials

### Immobilized Heparin Beads (Pierce)

1. 20 mM potassium phosphate buffer, pH 7.4.

2. 1.5 mL centrifuge tubes.

3. A rotator

### Procedure

Dilute 25 μL sera samples with 500 μL of 20 mM potassium phosphate buffer, pH 7.4. Mix and put on ice until use.

4. Pipette 50 μL slurry of heparin beads into a 1.5 mL tube.

5. Wash the beads once with 500 μL water, 3 times with 500 μL of the phosphate buffer (spin down the beads and remove the buffer between washes).

6. Add the sample to the beads and incubate with rotating in cold room for 30 min.

7. Spin and remove the supernatant to another tube.

8. Wash the beads at least 3 times with 500 μL of the binding buffer.

9. Add 30 μL 2×samples buffer directly to the beads and boil for 5 min.

10. Spin, analyze the supernatant by 1D gel electrophoresis. Heparin-conjugated agarose beads were used as an affinity column to pull down all heparin-binding molecules in AD and age-matched control sera samples.

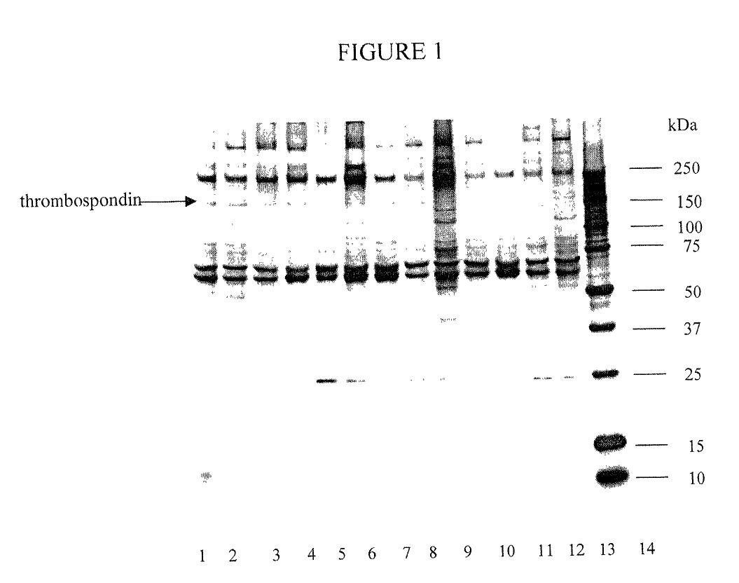

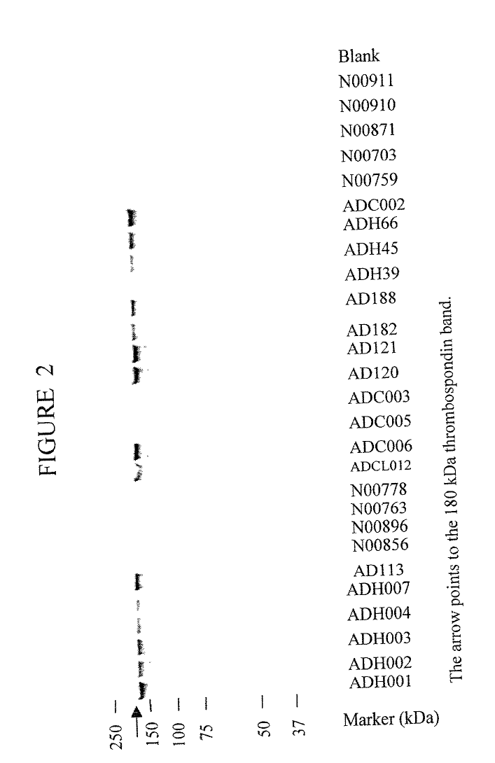

With reference to FIG. 1, an analysis of thrombospondin levels in human sera enriched by heparin affinity column is shown. The affinity column purified samples were run on a 10-20% precast tricine gel, supplier Invitrogen. The samples are: lane 1, AD120; lane 2, AD121; lane 3, AD182; lane 4, AD188; lane 5, ADH39; lane 6, ADH45; lane 7, ADH66; lane 8, ADC002; lane 9, N00759; lane 10, N00703; lane 11, N00871; lane 12, N00910; lane 13, N00911; lane 14, BioRad precision protein marker. (AD refers to Alzheimer's disease patients, whereas N refers to age-matched normal human sera). The arrow in FIG. 1 indicates a 180 kDa band that shows up in all AD samples and is not visible in most of the age-matched controls. This band in gel digested with trypsyn was sequenced by QSTAR Pulsar I (MDS Sciex) mass spectrometry. Five most intensive peaks of trypsyn peptide were sequenced and all of these match to thrombospondin. The arrow indicates the 180 kDa band that show up in all the AD samples and are not visible in 11 of the 15 age-matched controls. From FIG. 2 it can be seen that all 13 AD samples show positive, 11 of the normals show negative, 4 of the age-matched controls show negative with possible dementia.

Now referring to FIG. 2, a Western blot analysis of thrombospondin levels in human sera of Alzheimer's patients and age-matched control is illustrated.

2 μL of serum for each sample was run on 10-20% tricine gels. The gels were transferred onto nitrocellulose membrane and blocked with 5% skim milk, MBST (0.1% tween twenty). 1 μg/mL of mouse anti human TSP-1 monoclonal antibody (Thrombospondin-1, Ab-11, available from Lab Vision Corporation) in PBST containing 5% skim milk was used. The use of various antibody fragments is also contemplated. Goat anti mouse Ab conjugated with HRP was used as secondary Ab (1:4000 in PBST containing 5% skim milk). The arrow indicates the 180 kDa band that show up in all the AD samples and are not visible in 11 of the 15 age-matched controls. From FIG. 2 it can be seen that all 13 AD samples show positive, 11 of the normals show negative, 4 of the age-matched controls show negative with possible dementia. These results show that thrombospondin can be used as a marker for early diagnosis of Alzheimer's disease.

The markers which are analyzed according to the method of the invention are released into the circulation and may be present in the blood or in any blood product, for example plasma, serum, cytolyzed blood, e.g. by treatment with hypotonic buffer or detergents and dilutions and preparations thereof, and other body fluids, e.g. CSF, saliva, urine, lymph, and the like. In another preferred embodiment the presence of the markers in CSF may be measured.

Senile plaque-dense regions of the brain of patients with AD represent environments of elevated oxidative stress and that protein in the brain of patients with AD is more oxidized than that of controls. Reactive microglia extensively present with senile plaque regions have been proposed as a source of oxyradicals in the brain.

In a further contemplated embodiment of the invention, body fluid samples may be taken from a patient at one point in time or at different points in time for ongoing analysis. Typically, a first sample is taken from a patient upon presentation with possible symptoms of dementia and analyzed according to the invention. Subsequently, some period of time after presentation, for example, about 3-6 months after the first presentation, a second sample is taken and analyzed according to the invention. The data can be used to diagnose AD, rule out AD, or distinguish between AD and non-AD dementia. By “sample” is meant a body fluid such as blood, CSF, urine, salvia, and the like.

The presence of thrombospondin is determined using antibodies specific therefor and detecting specific binding of the antibody to its respective marker. Any suitable direct or indirect assay method may be used, including those which are commercially available to determine the level of the thrombospondin measured according to the invention. The assays may be competitive assays, sandwich assays, and the label may be selected from the group of well-known labels such as radioimmunoassay, fluorescent or chemiluminescence immunoassay, or immunoPCR technology. Extensive discussion of the known immunoassay techniques is not required here since these are known to those of skilled in the art. See Takahashi et al. (Clin Chem 1999;45(8):1307) for S100B assay.

Although not limited thereto, the immunoassay method used in the instant examples may comprise a double antibody or sandwich ELISA for measuring the level of thrombospondin in the sample. According to this method, one of the antibodies is a “capture” antibody which is immobilized onto a solid-phase, and the other is a “detector” antibody which is labeled with, for example, an enzyme. The detector antibody binds to marker protein bound to the capture antibody to form a sandwich structure. A marker protein standard is used to prepare a standard or calibration curve of absorbance vs. marker protein concentration.

The assay method used to measure the presence of thrombospondin should exhibit sufficient sensitivity to be able to measure each protein over a concentration range from normal values found in healthy persons to elevated levels in people evidencing disease, i.e. 2SD above normal (=cut-off) and higher.

The assay may be carried out in various formats, including a microtiter plate format which is preferred for carrying out assays in a batch mode. The assays may also be carried out in automated analyzers, such as those maintained at central laboratories, which are well known in the art. Another assay format which can be used according to the invention is a rapid manual test which can be administered at the point-of-care at any location. Typically, such devices will provide a result which is above or below a cut-off, i.e. a semiquantitative result.

The protein, thrombospondin, of the present invention may be used in any immunoassay system known in the art including, but not limited to: radioimmunoassay, enzyme-linked immunosorbent assay (ELISA), “sandwich” assays, precipitin reactions, gel diffusion immunodiffusion assay, agglutination assay, fluorescent immunoassays, protein A or G immunoassays and immunoelectrophoresis assays. According to the present invention, monoclonal or polyclonal antibodies produced against thrombospondin are useful in an immunoassay on samples of blood or blood products such as serum, plasma or the like, spinal fluid or other body fluid, e.g. saliva, urine, lymph, and the like, to diagnose patients with dementia, particularly AD.

The antibodies can be used in any type of immunoassay. This includes both the two-site sandwich assay and the single site immunoassay of the non-competitive type, as well as in traditional competitive binding assays. Alternatively, thrombospondin may be used in a suitable assay whose goal is to determine the presence of thrombospondin autoantibodies.

Particularly preferred, for ease and simplicity of detection, and its quantitative nature, is the sandwich or double antibody assay of which a number of variations exist, all of which are contemplated by the present invention. For example, in a typical sandwich assay, unlabeled antibody is immobilized on a solid phase, e.g. microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which may be quantitated by comparison with control samples containing known amounts of antigen.

In summary, the inventive concept is drawn toward a process for the determination of dementia, particularly MCI or Alzheimer's dementia, according to the principle of immunoassay, characterized in that a serum or plasma sample with at least one antibody against thrombospondin and a binding partner for thrombospondin or for the antibody is incubated, whereby either the antibody against thrombospondin or the binding partner is labeled with a determinable group, the thereby formed immunological complex which contains the determinable group is separated off and the determinable group in the separated off or still remaining phase is determined as measure for thrombospondin from the sample. The process may be further characterized in that the sample with an antibody against thrombospondin and a conjugate from an antibody against thrombospondin and a determinable group is incubated, the formed immunological complex is separated by phase separation and the determinable group is determined in one of the phases; alternatively, a sample with an antibody against thrombospondin and a conjugate of thrombospondin and a determinable group is incubated, the formed immunological complex is separated off by phase separation and the determinable group is determined in one of the phases.

In its broadest context, the invention is directed toward the use of antibodies against thrombospondin or autoantibodies against thrombospondin antibodies for the determination of dementia, particularly MCI or Alzheimer's dementia.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

One skilled in the art will readily appreciate that the present invention is well adapted to carry out the objects and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for diagnosing dementia in a mammal comprising:
obtaining a sample of body fluid from said mammal;
contacting said sample with at least one antibody which specifically binds to a marker indicative of thrombospondin;
determining a presence of thrombospondin in said sample; and
correlating the presence of thrombospondin with the occurrence of dementia.

2. The method for diagnosing dementia in a mammal, in accordance with claim 1 wherein:
said dementia is Alzheimer's dementia.

3. A method as in claim 1, wherein said sample of body fluid is blood or any blood product.

4. A method as in claim 1, wherein said measuring of said sample is by an immunoassay technique.

5. A diagnostic kit for diagnosing and monitoring the progression of dementia comprising:
at least one antibody which is specific for a marker indicative of thrombospondin, said antibody or marker capable of being immobilized on a solid support;
and at least one labeled antibody, which binds to said marker;
whereby at least one analysis to determine a presence of a marker, an antibody specific thereto, or their immunologically detectable fragments, is carried out on a sample of a body fluid.

6. A kit as in claim 5 wherein said sample of body fluid is blood or a blood product.

7. A kit as in claim 5 wherein diagnosing and monitoring is carried out on a single sample of body fluid.

8. A kit as in claim 5 wherein diagnosing and monitoring is carried out on multiple samples of body fluid; such that at least one analysis is carried out on a first sample of body fluid and at least another analysis is carried out on a second sample of body fluid.

9. A method as in claim 8 wherein said first and second samples of body fluid are obtained at different time periods.

10. A process for the determination of dementia according to the principle of immunoassay, characterized in that a serum or plasma sample with at least one antibody against thrombospondin and a binding partner for thrombospondin or for the antibody is incubated, whereby either the antibody against thrombospondin or the binding partner is labeled with a determinable group, the thereby formed immunological complex which contains the determinable group is separated off and the determinable group in the separated off or still remaining phase is determined as measure for thrombospondin from the sample.

11. A process according to claim 10 characterized in that the sample with an antibody against thrombospondin and a conjugate from an antibody against thrombospondin and a determinable group is incubated, the formed immunological complex is separated by phase separation and the determinable group is determined in one of the phases.

12. A process according to 10 characterized in that the sample with an antibody against thrombospondin and a conjugate of thrombospondin and a determinable group is incubated, the formed immunological complex is separated off by phase separation and the determinable group is determined in one of the phases.

13. Use of antibodies against thrombospondin for the determination of dementia.

14. Use of autoantibodies against thrombospondin antibodies for the determination of dementia.
